(19) United States
(12) Patent Application Publication
(10) Pub. No.: US 2026/0194863 A1
(43) Pub. Date: Jul. 9, 2026

(54) TIMING DEVICE

(71) Applicant: Big Potato Enterprises Limited, London (GB)

(72) Inventor: Gary Pyper, London (GB)

(73) Assignee: Big Potato Enterprises Limited, London (GB)

(21) Appl. No.: 19/442,523

(22) Filed: Jan. 7, 2026

(30) Foreign Application Priority Data
Jan. 8, 2025 (EE) P202500002

(51) Int. Cl. G04F 1/04; A63F 11/00

(52) U.S. Cl. CPC G04F 1/04; A63F 11/00; A63F 2250/1063

(57) ABSTRACT

A timing device includes a ball and a platform. The platform has a path forming a zig zag route from side to side of the platform between the ends of the platform, and a point along the path where the ball can be introduced. The ball may drop out of the path at holes at each of the two ends of the path. A stand is configured to support the platform, allowing the path to pivot around a central line, where each end of the path is able to be tilted upwards or downwards to cause the ball to roll along the path towards the end that is tilted downwards in a predetermined time period.

### CROSS-REFERENCE TO RELATED APPLICATIONS

This application is a non-provisional patent application which claims priority to Estonian Patent Application No. 70502 filed Jan. 8, 2025, and entitled “Timing Device”, which is hereby incorporated herein by reference in its entirety.

### BACKGROUND

The present disclosure relates to a timing device and related methods and in particular embodiments to a timing device where a ball rolling down a path which can be tilted up or down at each end is used to keep time.

Timers such as those disclosed in U.S. Pat. No. 5,005,160A, 3,101,587A may play an important role in board games and other toys. They have applications such as allowing timed segments to be measured in games. These timers need to be able to measure a specific period of time for each segment, and provide a visual or audible indicator of how much time is left until the timer runs out, and when the timer has ended. It may furthermore be required to be able to adjust the time for each segment based on factors such as the time spent on the previous segment, or the skill of each individual player. Many existing timers can only time a specific set time period, and are unable to adjust the time for a specific segment, based on the time of the previous segment or segments. Additionally, existing non-electric timers are generally not configurable to allow different segments to be timed for different periods using the same piece of timing apparatus. Electric timers may more easily allow for different time periods, but are typically more complicated and expensive to manufacture. This may provide a limitation to their use in games and toys.

The present application aims to address some or all of these limitations.

### SUMMARY

According to a first aspect of the present disclosure there is provided a timing device comprising, a ball; a platform, comprising a path forming a zig zag route from side to side of the platform between the ends of the platform, a point along the path where the ball can be introduced, and holes at each of the two ends of the path, through which the ball may drop out of the path, and; a stand arranged to support the platform, allowing the path to pivot around the central line, such that the path is able to be tilted upwards or downwards at each end, so that the ball will roll along the path towards the end which is angled downwards in a predetermined time period.

In prior art apparatus for timing short time periods, typically an hourglass set up would be used, where the inverting of the hourglass would start the timer. In these designs the sand running into the bottom section of the hourglass would signify the passage of time, and the timer would end when the sand from the top portion had fallen completely into the bottom section. In the present disclosure, the small ball running along the path presents a clearer visual and audible indication of the time that has been expended, and the ball falling out of the hole at the end of the path, presents a clearer visual and audible end of the time period.

An hourglass timer can be inverted at any time, to restart the timer, either when the sand has fully filled the lower chamber or when the timer is still running. Similarly, in the present disclosure the timer can be tilted, to invert the timer, and make the ball roll in the other direction. An advantage of embodiments of the present disclosure over the hourglass design, is that the base supporting the enclosed path is much wider than the base of a typical hourglass timer for short time periods, such as is commonly seen in board games. This gives the timer greater stability, making it much harder to knock over, especially when the timer is inverted quickly.

In an embodiment, the platform comprises two sections; a base section with a path along which the ball will roll, and a top section, with a hole at the midpoint for introducing the ball. The advantage of the top section is to ensure that the ball does not fall out of the path as it rolls along. Dropping the ball into the hole at the midpoint provides a clear start point for the timer. The two holes in the bottom section of the path, and both equidistant from the midpoint of the path, ensuring that the ball will take equal amounts of time to roll along the path from the midpoint in either direction, provided the path is tilted to the same angle. Alternatively, holes in the top section at various points down the path, as opposed to just the midpoint, could be used to allow the timer to be used to measure different time periods.

In an embodiment, the top section of the path is comprised of transparent material. This allows the passage of the ball along the path to be clearly visible.

In an embodiment, the platform is planar and the hole at the midpoint of the top section of the platform is equidistant from the two holes at the ends of the path in the base section, thus ensuring that the ball takes an equal length of time to roll from the midpoint to either of the two outside holes, when the path is tilted at the same angle. This allows the timer to be used to time successive turns within a game for the same length of time, by placing the ball into the path at the midpoint at the start of each turn.

In an embodiment, the top and bottom sections are joined together with irreversible fixtures. This is to ensure ease of manufacturing, such that the top and bottom sections of the path may be manufactured separately, and subsequently joined together.

In an embodiment the path is formed of interconnecting diagonal passageways, each at an angle to the central line between 1 and 25°, such that adjacent passageways are angled in opposite directions and are connected by curved portions. This structure allows the ball to pass smoothly along the path, and provides an audible indication of the ball travelling along the path, as the sound of the ball rolling will change as it goes around each corner.

In an embodiment the platform has two rods extending outwards from the sides, along the central line, about which it pivots. In another embodiment the stand is comprised of two upwards extending supports, each having a cutout or hole in the top to accommodate the two rods extending outwards from the path, allowing these to act as pivot points. This provides a more stable pivot mechanism for the path, compared with the use of a single central fulcrum.

In an embodiment the stand has a base having two recessed sections situated at the ends of the base, beneath the respective holes at the ends of the path, arranged to receive the balls when they drop out of the path. This provides a space for the ball to fall into once it has reached the end of the path and fallen out of the hole. This ensures that the ball does not roll away and become lost. Furthermore, when the ball falls into this recessed section, it provides an audible as well as visual indication that the time has ended.

In an embodiment, the two upright supports, are removable from the stand base. In another embodiment, the path is further removable form the upright supports, by removing the outwards extending rods, attached to the platform, from the cutouts, or holes. These features allow the timing device to be disassembled and thus packed flat into a small space such as a game box. This makes the device more convenient for storage purposes than prior art examples of timing devices which are generally not able to be disassembled.

In an embodiment the base at least one recessed area, each side of the central line, each of which can accommodate an upwards standing peg, which prevent the platform from pivoting beyond a set angle. In another embodiment, the pegs can be placed on either side of the midpoint line in any one of multiple recessed areas, such that the maximum angle of rotation of the platform either to the left or right of the midpoint is dictated by the placement of the pegs.

Timers which exist in the prior art, such as hourglasses, typically have the limitation that they are only able to measure a predetermined time period. In an hourglass example this would correspond to the time taken for the sand to pass from one chamber to the other. While it is possible to invert the timer before all of the sand has passed through, it is not typically possible to set up the timer such that different lengths of time can be measured each time the timer is used. The advantage of the pegs described above is that they can be used to act as a support for the enclosed path when it is tilted towards an individual peg. This acts to prevent the enclosed path tilting beyond a certain angle. If one of the pegs is moved into an area further from the midpoint of the base, the angle to which the enclosed path can tilt in that direction is decreased, if the peg is moved closer to the midpoint the angle to which the enclosed path can tilt is increased. This in turn, affects the speed at which the ball can roll down the path. If the path is tilted at a greater angle the ball will pass along the path at a greater speed. Therefore, the timer described in the present disclosure has the advantage over the prior art, that it can be used to measure different time periods when the path is tilted in one direction compared to the other, and that the time period measured when the path is tilted in either direction can be easily changed. This can be useful in gameplay in providing a handicap system, for example the pegs could also be used to keep score, and simultaneously handicap the person winning, thereby leading to a more competitive game.

In an embodiment the path and base of the timing device may both be manufactured from moulded plastic. This allows the device to be manufactured quickly and at low cost, this provides a significant advantage over electronic timing devices, which are generally costly to manufacture.

In an embodiment there is defined a game comprising the timing device described above, used to time a turn in the game by tilting one end of the platform downwards and placing the ball into the path at the midpoint, the turn ending either when the ball drops out of the hole at the end of the path, or when the platform is tilted downwards at the opposite end. This allows for successive game turns to be timed in quick succession, with each player tilting the timing device towards them at the end of their turn. This can continue until the ball falls out of the path during one of the player's turns. This could signify the end of a round in the game.

In an embodiment, there is defined a method of timing a time period with the timing device described above, comprising, setting the angle of a platform, introducing a ball into a path and waiting for it to travel down the path.

It will be appreciated that any features expressed herein as being provided “in one example” or “in an embodiment” or as being “preferable” may be provided in combination with any one or more other such features together with any one or more of the aspects of the present disclosure.

### BRIEF DESCRIPTION OF DRAWINGS

Embodiments of the present disclosure will now be described by way of example with reference to the accompanying drawings, in which:

FIG. 1 shows a disassembled view of an example a timing device according to an embodiment of the present disclosure;

FIG. 2 shows a side view of the assembled timing device;

FIG. 3 shows an end view of the timing device;

FIG. 4 shows a view of the timing device from above; and

FIG. 5 shows a cross section of the timing device from a side view.

### DETAILED DESCRIPTION

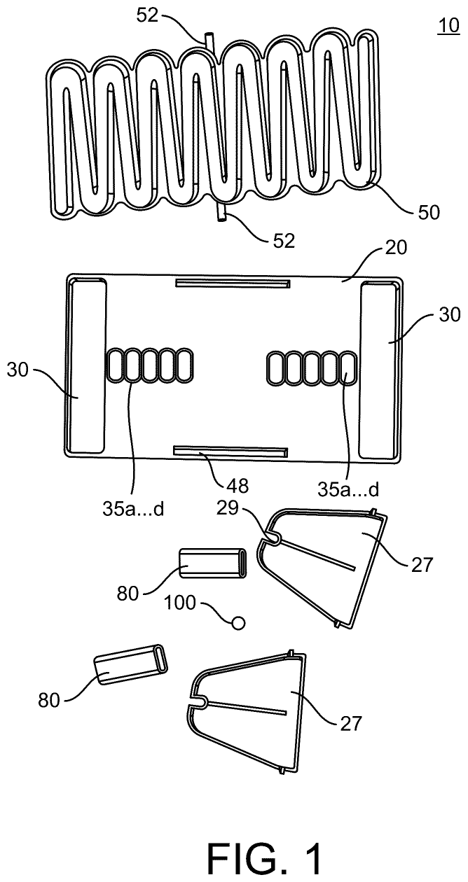

FIG. 1 shows the components of a timing device 10 disassembled. The device comprises a platform 50 that includes an internal path 53. A ball 100 is provided to run along the path 53 contained within the platform. A base section 20 is provided to support the platform and allow it to pivot about two rods 52 extending outwards from the central line 65 of the platform.

The base section 20 has two recessed sections 48 along the remining two parallel sides, designed to receive two upwards standing supports 27. The base section is designed to stand stably on a flat table surface or other support surface. The base section 20 also has a series plural recessed sections 35 (a, b, c, d) extending towards the central line 65 on ach side, arranged to accommodate two upwards standing pegs 80, each received in one of the recessed sections 35 on each side. The base section further comprises a recessed section 30 at either end to receive the ball 100 when it drops out of the platform.

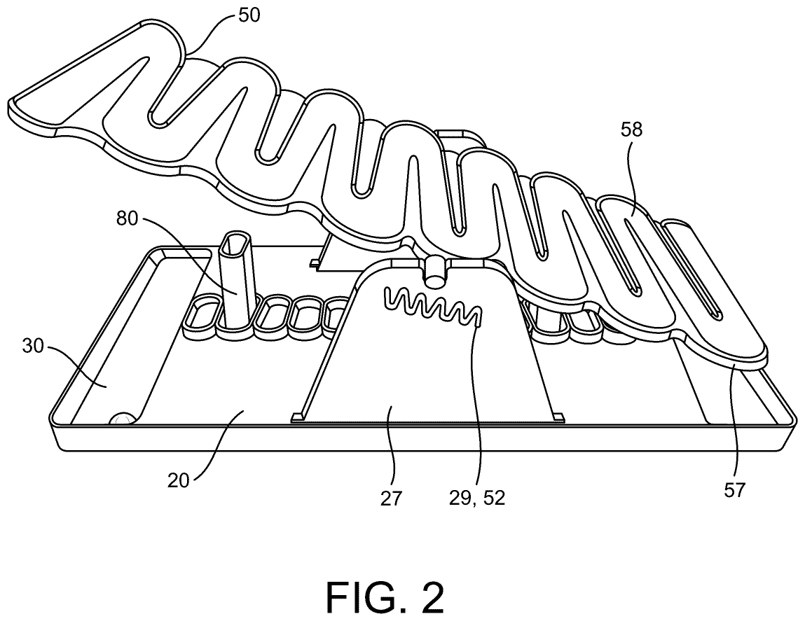

The platform is shown more clearly in FIGS. 2-5, to comprise two sections, a base section 57 with a planar path 53 along which the ball can roll, and a top section 58 which fits together with the base section by means of irreversible fixtures, such as adhesive, welding, snap fit, etc. At least the base section comprises a planar path formed therein which forms a zig zag route of interconnecting diagonal passageways from side to side of the platform, each at an angle to the central line 65, such that adjacent passageways are angled in opposite directions and connected by curved portions. Generally, a shallow angle/slope to the zig zag is preferred to control the rate at which the ball falls, e.g., between 1 and 25°. At each of the two ends of the path there is a hole in the base, out through which the ball drops when it reaches the end signifying the end of the timed period. As described above, the base has recessed portion to catch the ball when it drops. In other examples, the ball may be caught by other means, such as a magnet in the base (where the ball is a metal bearing), etc.

The top section of the platform encloses the path, ensuring the ball cannot fall out until it reaches the end of the path. The top section 58 may also have the angled path formed in it such that the base section and top section in combination form a tube along which the ball runs. This top section has a hole 56 at the midpoint of the path, i.e., coinciding with the pivot line, equidistant from the two holes 54a,b in the base of the platform. Therefore, the distance the ball travels from the midpoint to either of the two holes at the endpoints of the path is equal, thereby ensuring that the time taken for the ball to travel down the path is the same in either direction provided the platform is tilted at the same angle.

Preferably the top section of the path is comprised of transparent plastic to allow the path of the ball to be clearly visible. The top 58 and bottom 57 sections of the platform can be manufactured together or separately and then joined.

In other examples the platform could comprise only the base section of the platform, without the need for a top section. The insertion point at which the ball is introduced to the path, e.g., the midpoint 56, could in this instance be marked on the base 57 section of the platform. In other examples, different insertion points could be provided, e.g., away from the midline, to provide for different or additional time periods.

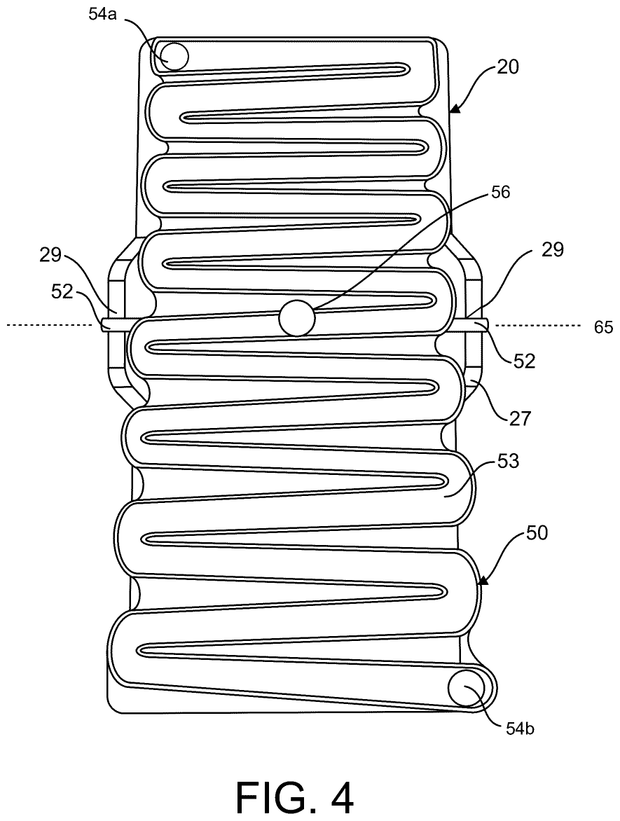

Additionally, while in FIG. 4 the path within the platform is shown comprising 15 interconnecting diagonal sections, in other designs this path could comprise more or fewer sections, e.g., 11, 13, 17 or 19, provided the midpoint is located in the centre of the middle diagonal section. Similarly, the path may be sinusoidal or have other forms that run from side to side of the platform multiple times such that the overall path extends from one end of the platform to the other.

The fully constructed timing device is shown more clearly in FIG. 2. The base section 20 and the upright supports 27, may be slotted together by fitting the upright supports 27 into the recessed sections 48 along the sides of the base. These can click or friction fit into place and stand upright. The two upright supports 27 contain holes or cutouts 29 in the tops. The two rods 52 extending from the sides of the platform 50, are designed to fit snugly into the cutouts or holes 29, such that the platform 50 is able to pivot around the central line 65 of the device. Other designs for the platform and support may be used, provided that the platform is able to pivot, with each end able to be tilted upwards or downwards, to allow the ball to roll down the path towards whichever end is lowermost. For example, the two upright supports could have inwards extending rods, configured to clip onto cutouts or holes in the side of the path, or other arrangements are possible.

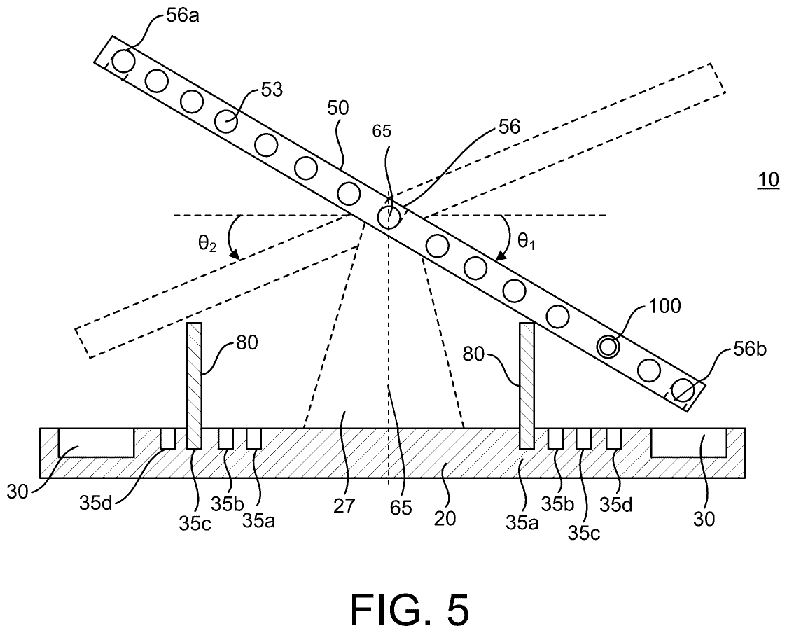

When the device is fully constructed, as shown in any of FIGS. 2-5, the platform 50 is suspended above the stand base, held in place by rods 52 clipped or slid into the cutouts or holes in the upright supports. The platform is able to pivot around the central line 65, which runs between the two supports and the midpoint hole 56 through which the ball enters the path. FIG. 5 shows a diagram of the platform pivoting around the centre line. The angle at which the platform is pivoted, it hereafter defined as θ₁ which is the angle between the lower side of the platform, and the line from the midpoint running parallel to the base. The base has stops, in this case moveable pegs 80 described in more detail below, to limit the degree to which the platform can be tilted down in each direction.

When the platform is pivoted to an angle θ₁, the ball will run down the path from the midpoint through which it entered the path until it falls out of the hole at the end of the path into the recessed potions in the base in a particular time period. The ball will be visible running down the path through the transparent top portion of the platform, and the ball will also make a noise as it travels along the path, and around the corners. The timer ends when the ball falls out of one of the holes 54a,b at the ends of the path, into the recessed sections 30 in the base situated directly below the holes, making a noise as it lands.

The timer is designed for use in a game to time players turns, an example is as follows: At the start of the game the ball can be placed into the platform 50 at the midpoint 56 of the path 53. When the first player begins their turn, they will tilt the platform towards them at an angle θ₁ and the ball will begin rolling towards the hole at the end of the path closest to them. They will attempt to complete their turn before the ball reaches the end of the path and falls out. If they complete their turn before the ball falls out, the next player's turn commences and the second player will tilt the platform towards then, such that the ball is now running in the opposite direction. The second player again tries to complete their turn before the ball reaches their end of the path and falls out of the hole. This repeats with each player taking their turn, until one player fails to complete their turn before the timer ends, i.e., the ball falls out of the path. The timer ending signifies either the end of a round or the end of the game. The next round can be started by placing the ball back in the path via the midpoint hole.

In this example the length of each player's turn depends on how far along the full length of the path the ball was able to roll during the previous player's turn. Thus, if it takes time T for the ball to roll from the midpoint to the end with the platform at a particular angle, the initial player has time T to complete their turn and pivot the platform. Where this takes time t1, the next player has t1+T to complete their turn (assuming the platform is tilted to the same angle in both directions). Where the second player's turn takes time t2, the first player has t2−t1+T to complete their turn, and so on. The turns continue until a player cumulatively takes more than time T than their opponent at which point the ball reaches the end of the path and drops out, ending that round of the game.

The timer could also be used to time each player's turn for the same period of time, by allowing the ball to fall out of the path at the end of each turn, and then introducing it back to the path at the midpoint at the start of the next player's turn.

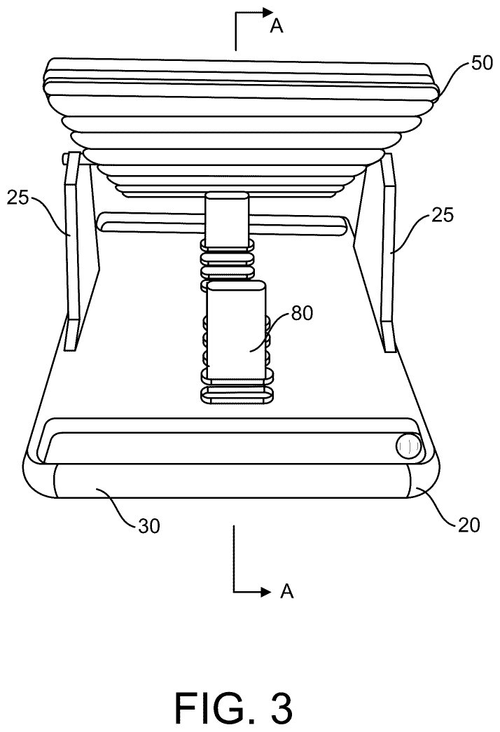

FIG. 3 shows the base of the timing device which contains multiple recessed areas 35a,b,c,d, either side of the central line 65, each of which can accommodate an upwards standing peg 80, which acts as a stop and prevent the platform from pivoting beyond a set angle θ₁. These can be placed on either side of the midpoint line in any one of multiple recessed areas, such that the maximum angle of rotation θ₁ of the platform either to the left or right of the midpoint is dictated by the placement of the pegs 80.

As shown in FIG. 5, if one of the pegs is moved into an area further from the midpoint of the base, the angle θ₂ to which the enclosed path can tilt in that direction is decreased. If the peg is moved closer to the midpoint, the angle θ₁ to which the enclosed path can tilt is increased. This in turn, affects the speed at which the ball can roll down the path. If the path is tilted at a greater angle the ball will pass along the path at a greater speed and thus over a shorter time period. Thus, the amount of time available to the players for their turns can be altered by moving both pegs either inwards or outwards.

Furthermore, this can be useful in gameplay in providing a handicap system by having the pegs at different positions on each side thus leading to the ball rolling at different rates for the two players/teams. In one example, the pegs could also be used to keep score, and simultaneously handicap the person winning, thereby leading to a more competitive game. In the example gameplay previously described, the players tilt the timing device towards them at the start of each turn, such that a round ends when the ball falls out of the path. In this example the pegs could be used at the end of each round, to give the player who won the previous round a handicap by moving the peg on the side of the base the winner was sitting on could be moved closer to the midpoint line. During the next round the player who had won the previous round would thus have the ball travelling faster along the path towards then during each turn, giving them less time to complete their turn.

The timing device can be made of a material with low manufacturing costs such as moulded plastics, and the ball can be made of a material such as a glass marble or metal bearing, which is heavy enough to run down the path without undue resistance.

Embodiments of the present disclosure have been described with particular reference to the examples illustrated. However, it will be appreciated that variations and modifications may be made to the examples described within the scope of the present claims.

1. A timing device, comprising:
a ball;
a platform, comprising a path forming a zig zag route from a first side of the platform to a second side of the platform between ends of the platform, wherein the path includes:
a point at which the ball can be introduced;
a first hole at a first end of the path; and
a second hole at a second end of the path, wherein the ball is configured to drop out of the path through the first hole or the second hole; and
a stand configured to support the platform to allow the path to pivot around a central line, wherein each end of the path is able to be tilted upwards or downwards to cause the ball to roll along the path towards the end that is tilted downwards in a predetermined time period.

2. The timing device of claim 1, wherein the platform comprises:
a base section including the path along which the ball is configured to roll; and
a top section including a third hole at a midpoint, wherein the third hole is configured to receive the ball and introduce the ball to the path.

3. The timing device of claim 2, wherein the top section comprises a transparent material.

4. The timing device of claim 2, wherein the platform is planar and the third hole is equidistant from the first hole and the second hole, such that the ball takes an equal amount of time to roll from the midpoint to the first hole as to roll from the midpoint to the second hole when the path is tilted at a same angle toward the first end and the second end, respectively.

5. The timing device of claim 1, wherein the path is formed of interconnecting diagonal passageways, each at an angle to the central line between 1° and 25°, such that adjacent passageways are angled in opposite directions and are connected by curved portions.

6. The timing device of claim 1, wherein the platform comprises two rods extending outwards from the sides of the platform, along the central line, about which the platform pivots,
wherein the stand comprises two upward extending supports, each having a cutout or hole to accommodate one of the two rods such that the cutouts or holes of the supports act as pivot points,
wherein the two supports are removable from a base of the stand, and
wherein the platform is removable from the supports by removing the rods from the cutouts or holes.

7. The timing device of claim 1, wherein the stand comprises a base having two recessed sections situated at opposite ends of the base, beneath the first and second holes, respectively, wherein the recessed sections are configured to receive the ball when the ball drops out of the path.

8. The timing device of claim 1, wherein the base comprises at least one recessed area on each side of the central line, wherein each recessed area is configured to accommodate an upwards standing peg to prevent the path from pivoting beyond a set angle.

9. The timing device of claim 8, wherein the pegs are configured to be placed on either side of the midpoint line in any one of multiple recessed areas, such that the maximum angle of rotation of the platform to either side of the midpoint is adjustable based on the placement of the pegs.

10. The timing device of claim 1, wherein the platform and/or base are manufactured from a molded plastic material.

11. A game comprising the timing device of claim 1, wherein turn in the game is timed by tilting one end of the platform downwards and placing the ball into the path at the midpoint, and wherein the turn ends either when the ball drops out of the first or second hole, or when the platform is tilted downwards at an opposite end.

12. A method of timing a time period with the timing device of claim 1, comprising;
setting an angle of a platform;
introducing a ball into a path on the platform; and
waiting for the ball to travel down the path to an end of the path.
